(19) United States
(12) Patent Application Publication
(10) Pub. No.: US 2006/0140335 A1
(43) Pub. Date: Jun. 29, 2006

(54) System and method for helical cone-beam computed tomography with exact reconstruction

(76) Inventors: Dominic J. Heuscher, Aurora, OH (US); Frederic N. F. Noo, Salt Lake City, UT (US); Kevin M. Brown, Mentor-on-the-Lake, OH (US); Jed D. Pack, Salt Lake City, UT (US)

Correspondence Address: PHILIPS INTELLECTUAL PROPERTY & STANDARDS, 595 MINER ROAD, CLEVELAND, OH, 44143, US

(21) Appl. No.: 10/545,265

(22) PCT Filed: Feb. 9, 2004
(86) PCT No.: PCT/IB04/00386

§ 371 Date: Aug. 10, 2005

Related U.S. Application Data
(60) Provisional application No. 60/447,426, filed on Feb. 14, 2003.
(60) Provisional application No. 60/482,380, filed on Jun. 25, 2003.

(51) Int. Cl. H05G 1/60; A61B 6/00; G01N 23/00; G21K 1/12

(52) U.S. Cl. 378/4; 378/901

(57) ABSTRACT

A helical conebeam computed tomography imaging system includes an x-ray source (12) that produces an x ray conebeam, and an x-ray detector array (16) that detects the x ray conebeam after passing through an examination region (14). The x-ray detector array (16) generates projection data in a detector coordinate system defined with reference to the detector array (16). A derivative processor (60) computes a derivative of the projection data with respect to a helix angle of the helical trajectory at fixed projection direction to generate differentiated projection data. A convolution processor (64) convolves the differentiated projection data with a kernel function to produce filtered projection data. The convolving is performed in the detector coordinate system. A backprojector (42, 82) backprojects the filtered projection data to obtain an image representation.

The following relates to the diagnostic imaging arts. It finds particular application in helical conebeam computed tomography imaging, and will be described with particular reference thereto. However, it also finds application in other types of tomographic imaging.

Single-slice x-ray transmission computed tomography imaging systems employ limited beam dimensions, such as an essentially planar fan-beam that is rotated in a circular orbit around an imaging subject. The acquired projection data is reconstructed to generate an image slice. In multi-slice fan-beam imaging, data are acquired for a plurality of axially spaced-apart circular orbits, and reconstructed to produce a corresponding plurality of axially spaced-apart image slices. Multi-slice computed tomography imaging is conducive to fast and accurate image reconstruction due to the simplified fan-beam geometry. In one suitable reconstruction method, each slice is reconstructed separately using convolution backprojection. In another reconstruction method, the fan-beam projection data are rebinned into substantially parallel-ray views that are backprojected to reconstruct the image. Even in multi-slice methods, the rate of data acquisition is limited due to the small volume sampled by the essentially planar fan-beam and the discontinuous axially stepped orbiting of the x-ray source.

Helical conebeam computed tomography was developed to overcome these data acquisition rate constraints. A continuous helical x-ray source orbit is combined with a conebeam geometry that interacts with a three-dimensional volume to enable much higher data acquisition rates. A two-dimensional array of x-ray detector elements is arranged to receive the conebeam after it passes through an examination region in which the imaging subject is disposed. The detector array can be flat (flat detector geometry), curved (curved detector geometry), or otherwise shaped.

A disadvantage of helical conebeam computed tomography is that reconstruction of the conebeam projection data into an image representation is computationally complex. A given voxel of the image volume is sampled by non-coplanar projections spanning a portion of the helical trajectory of the x-ray source. In contrast, with the fan-beam geometry projections for a given voxel are substantially coplanar, that is, lie in a single plane.

Various conebeam reconstruction methods have been developed. Some of these techniques, such as the N-PI method, are not exact, in that they fail to accurately account for the cone angle. These inexact reconstruction methods introduce artifacts into the reconstructed image because they do not accurately account for the cone angles of the projections, that is, angular deviations of the projections from the axial plane. By employing various approximations relating to the cone angle, inexact reconstruction methods can provide rapid reconstruction with acceptably limited image artifacts for data acquired using small cone angles. However, these inexact reconstructed imnages become increasingly degraded by artifacts as the cone angle increases. Conebeams with large cone angles provide large beam interaction volumes and advantageously enable fast data acquisition.

For data acquired with large cone angles, an exact reconstruction method is preferably used that accurately accounts for projection components in the cone angle direction.

Prior exact reconstruction methods are more computationally intense than inexact methods due to the more complex conebeam geometry. An exact conebeam reconstruction method has been developed by Katsevich (see for example Katsevich et al, Proceedings SPIE Medical Imaging Conference, San Diego, Calif. (February 2003)).

However, this exact conebeam reconstruction method employs a voxel-based coordinate system that does not comport with conebeam data acquisition detector geometries.

Conversion of acquired projection data, which are defined using coordinates such as x-ray detector row and column values, projection angle coordinates, helix angle, or the like, into a detector-independent voxel-based coordinate system is computationally challenging and involves additional rebinning operations that substantially increase reconstruction time. Moreover, the previous exact conebeam reconstruction method is not readily adapted to existing backprojector processing pipelines such as are commonly employed in reconstructing parallel-rebinned projection data.

The present invention contemplates an improved apparatus and method that overcomes the aforementioned limitations and others.

According to one aspect, a conebeam computed tomography imaging system is disclosed. An x-ray source produces an x-ray conebeam directed into an examination region. The x-ray source is arranged to traverse a generally helical trajectory around the examination region.

An x-ray detector array is arranged to detect the x-ray conebeam after passing through the examination region. The x-ray detector array generates projection data in native scan coordinates defined with reference to the detector array. An exact reconstruction processor performs an exact reconstructing of conebeam projection data produced by the detector array into an image representation. The reconstructing is performed in the native scan coordinates

According to another aspect, a conebeam computed tomography imaging system is disclosed that produces conebeam computed tomography projection data having native scan coordinates that include at least a helix angle, a projection fan coordinate, and a projection cone coordinate. A means is provided for computing filtered projection data. The means for computing includes a differentiating means for computing a derivative of projection data with respect to the helix angle at fixed projection direction and a convolving means for convolving projection data with a kernel function. The convolving is performed in the native scan coordinates. A means is provided for backprojecting the filtered projection data to obtain an image representation.

According to another yet aspect, an exact reconstruction method is provided for reconstructing conebeam computed tomography projection data having native scan coordinates that include at least a helix angle, a projection fan coordinate, and a projection cone coordinate. Filtered projection data is computed by a combination of computing a derivative of projection data with respect to the helix angle at fixed projection direction and convolving projection data with a kernel function. The convolving is performed in the native scan coordinates. The filtered projection data is backprojected to obtain an image representation.

One advantage resides in fast, exact image reconstruction of conebeam imaging data acquired using a large cone angle.

Another advantage resides in simplified image reconstruction computations by obviating rebitiing operations associated with transforming tomographic projection data into a detector-independent voxel-based coordinate system.

Another advantage resides in optional incorporation of a backprojector pipeline configured for processing parallel-rebinned projection data in performing exact reconstruction of conebeam projection data.

Yet another advantage resides in straightforward accommodation of various detector geometries such as flat detector geometries and curved detector x-ray source-focused detector geometries.

Numerous additional advantages and benefits will become apparent to those of ordinary skill in the art upon reading the following detailed description of the preferred embodiments.

The invention may take form in various components and arrangements of components, and in various process operations and arrangements of process operations. The drawings are only for the purpose of illustrating preferred embodiments and are not to be construed as limiting the invention.

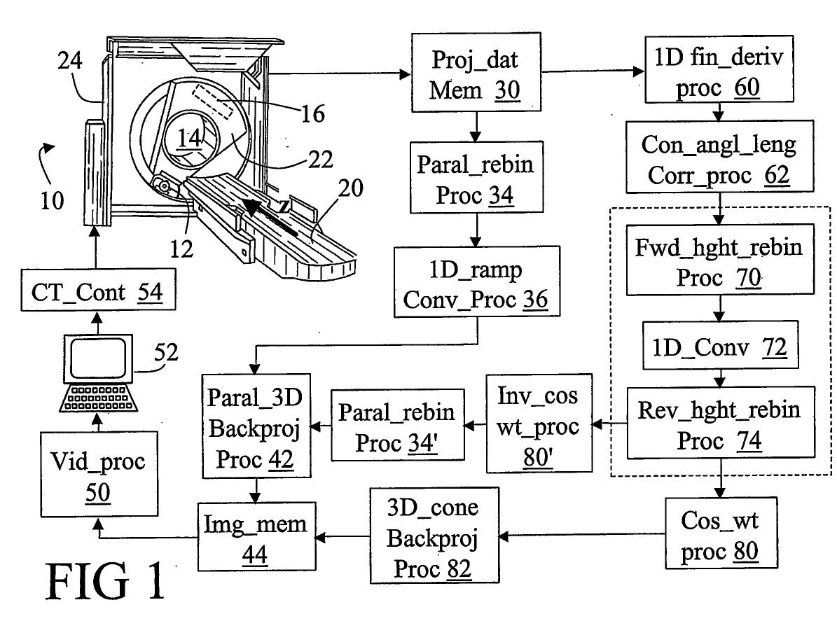

FIG. 1 diagrammatically shows a helical conebeam computed tomography imaging system including exact reconstruction of acquired conebeam projection data.

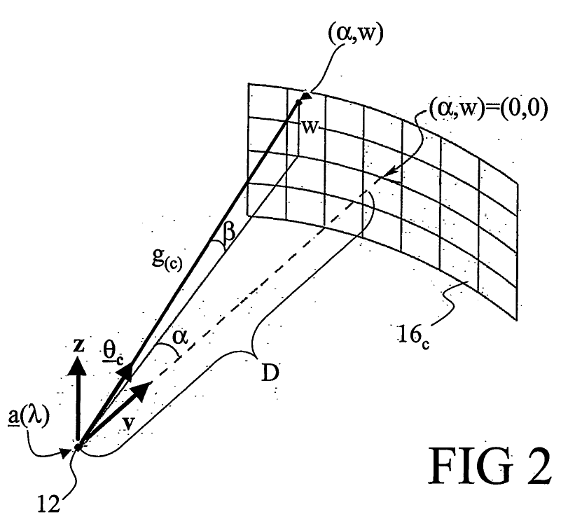

FIG. 2 diagrammatically shows a detector-based coordinate system for a source-focused curved detector geometry.

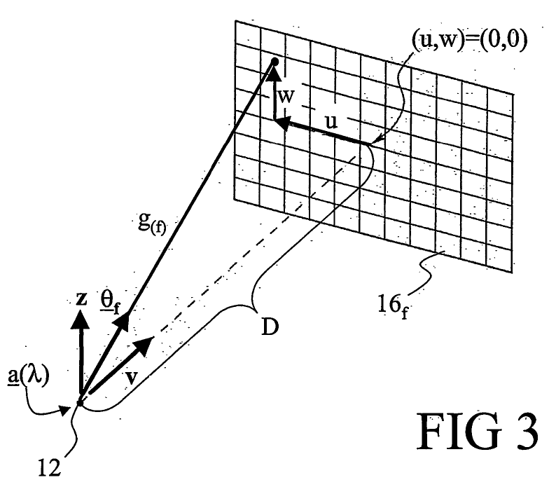

FIG. 3 diagrammatically shows a detector-based coordinate system for a flat detector geometry.

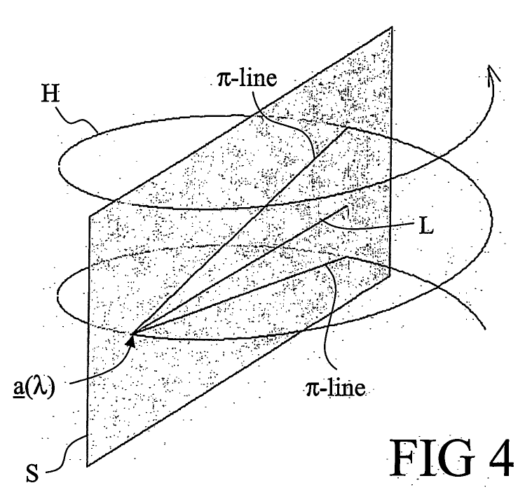

FIG. 4 diagrammatically shows geometric aspects related to π-lines and κ-planes.

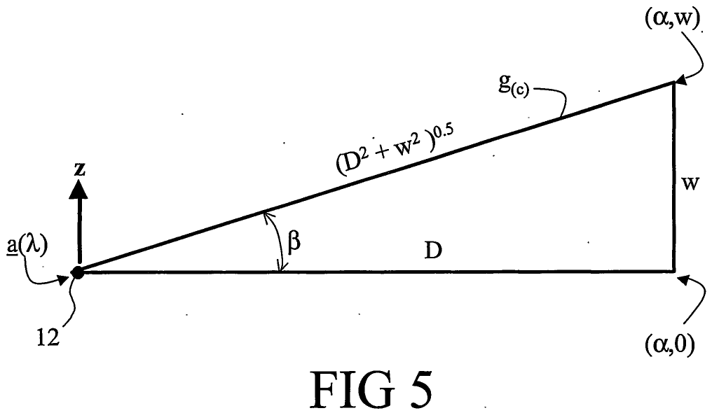

FIG. 5 diagrammatically shows the cos βcone angle length normalization for projection data in a source-focused curved detector geometry.

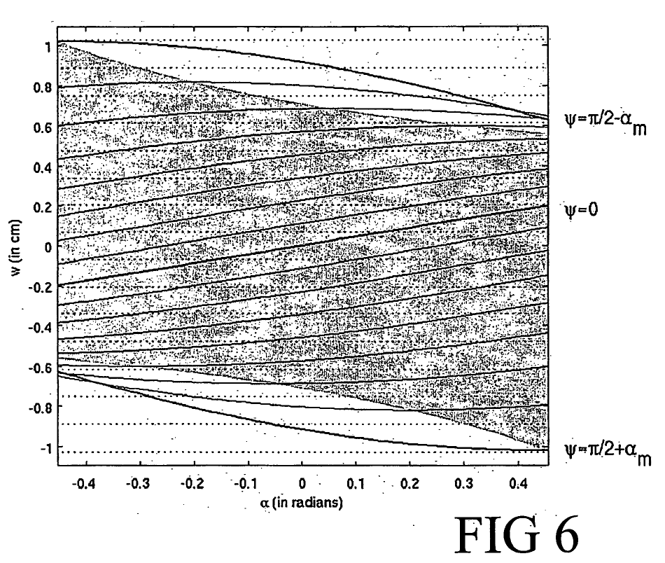

FIG. 6 shows exemplary κ-curves for a source-focused curved detector panel with sixteen detector rows, with a Tam-Danielsson window indicated by shading.

With reference to FIG. 1, a helical conebeam computed tomography imaging scanner 10 includes an x-ray source 12 that projects an x-ray conebeam into an examination region 14. After passing through the examination region, the x-ray conebeam is detected by a two-dimensional x-ray detector 16 (shown diagrammatically in phantom in FIG. 1) that includes an array of detector elements arranged to detect the x-ray conebeam after passing through the examination region 14.

To effect a helical trajectory of the x-ray source 12 about an imaging subject, the imaging subject is placed on a couch 20 or other support. The couch moves linearly along a z-direction as indicated. The x-ray source 12 and the x-ray detector 16 are oppositely mounted respective to the examination region 14 on a rotating gantry 22, such that rotation of the gantry 22 effects rotation of the x-ray source 12. Rotation of the gantry 22 along with simultaneous, continuous linear motion of the couch 20 effects a helical trajectory of the x-ray source 12 around the imaging subject disposed on the couch 20.

The x-ray detector 16 is shown mounted on the rotating gantry 22 such that it rotates along with the x-ray source 12 to intercept the x-ray conebeam throughout the helical trajectory. However, it is also contemplated to replace the x-ray detector 16 by an x-ray detector band mounted around a stationary gantry 24.

In operation, during helical orbiting of the x-ray source 12 relative to the imaging subject, the x-ray conebeam is projected into the examination region 14 where it interacts with the imaging subject. Some portion of the x-rays are absorbed by the imaging subject to produce a generally spatially varying attenuation of the x-ray conebeam. The x-ray detector 16 measures the x-ray intensities across the conebeam to generate x-ray absorption data that is stored in a projection data memory 30.

With continuing reference to FIG. 1 and with further reference to FIGS. 2 and 3, the detector 16 is preferably one of a source-focused curved detector 16_{c} shown in FIG. 2 and a flat detector 16_{f} shown in FIG. 3. The detector-based coordinate system of the projection data stored in the projection data memory 30 depends upon the detector geometry, such as the curved geometry of the source-focused curved detector 16_{c} shown in FIG. 2, or the flat detector geometry of the flat detector 16_{f} of FIG. 3. In both geometries, a detector-based coordinate v is directed from the x-ray source 12 toward a center of the x-ray detector 16. Native scan coordinates for the source-focused curved detector geometry and for the flat detector geometry are given in Table I.

TABLE I
Native Scan Coordinates Systems

| | Stationary | Flat | Curved |
|---|---|---|---|
| Parameter | coord. | detector coord. | detector coord. |
| In-slice | x | u | α |
| In-slice | y | v | v |
| Axial | z | w | w, β |
| Helix angle | — | λ | λ |

The (x,y,z) stationary coordinate system is a conventional Cartesian coordinate system and is provided for reference. The helix angle X identifies an angular position of the x-ray source 12 along the helical orbit.

With particular reference to FIG. 2, in the source-focused curved detector geometry a projection fan coordinate a indicates the projection angle in the fan angle direction, while the a projection cone angle coordinate β indicates the projection angle in the cone angle direction. A projection g_{(c)} lies along a projection direction vector θ_{c} in the curved detector geometry, and has coordinates g_{(c)}(λ, α, w) where w=D tan(β) and D is a source-to-detector distance from the x-ray source 12 to a center of the detector 16_{c}. The center of the curved detector 16_{c} is given by coordinates (λ, 0, 0), that is, α=w=0. The coordinate w is parallel to the axial or z-direction. The detector 16_{c} is not curved along the axial or z-direction, but is curved along the fan direction, that is, along the direction corresponding to the angle coordinate α. The detector curvature along the angle coordinate α is selected so that all detector elements for a given angle coordinate β are substantially equidistant from the x-ray source 12. That is, the detector curvature along the angle coordinate α is source-focused.

A vertex position A(λ) of the x-ray source 12 along the helical trajectory is given by helix angle λ for the curved detector geometry. In the stationary (x, y, z) coordinate system, the vertex points a(λ) of the helix, that is, the trajectory of the x-ray source 12, is given by:

$$\underline{a} ( λ ) = [ {R}_{o} cos ( λ + {λ}_{o} ) , {R}_{o} sin ( λ + {λ}_{o} ) , {z}_{o} + P \frac{λ}{2 π} ] \qquad ( 1 )$$

where Rₒ is a source distance, that is, a distance from the x-ray source 12 to the helix axis, P is the helical pitch, that is, the axial or z-distance the x-ray source moves relative to the imaging subject over a single helical trajectory turn, and λₒ and zₒ are a reference helix angle and reference axial position, respectively.

With particular reference to FIG. 3, in the flat detector coordinate system the coordinate u rotates with respect to the stationary x-coordinate during helical orbiting of the x-ray source. A projection g_{(f)} lies along a projection direction vector θ_{f} in the flat detector geometry, and has coordinates g_{(f)}(λ, u, w). (As noted previously, the symbol λ is used herein for the helix angle of the flat detector geometry, rather than the more conventional helix angle symbol θ). This projection is taken along a line from the x-ray source 12 toward detector coordinate (u, w) of the flat detector 16_{f}. The center of the flat detector 16, is given by coordinates (λ, 0, 0), that is, u=v=0.

The coordinate w is parallel. to the axial or z-direction. The source-to-detector distance D of the curved geometry is also defined for the flat detector geometry. Equation (1) specifying the vertex position a(λ) of the x-ray source 12 in the stationary (x,y,z) coordinate system also applies to the flat detector geometry of FIG. 3.

The detector arrays 16_{c}, 16_{f} are shown for convenience as having a small number of detector elements. In FIG. 2, the array of detector elements of the curved detector 16_{c} is represented by grid lines that indicate four rows of detectors along the axial or z-direction, with each row including seven detector elements. In FIG. 3, the array of detector elements of the flat detector 16_{f} is represented by grid lines that indicate eight rows of detectors along the axial or z-direction, with each row including eleven detector elements. Those skilled in the art will recognize that existing flat and source-focused curved detector arrays typically have much larger numbers of detector elements, such as a dozen or more rows of detectors, with each row including dozens or more preferably hundreds of detector elements. In one preferred embodiment, the detector has more than one hundred rows of 0.75 mm² detector elements.

With returning reference to FIG. 1, if an inexact image reconstruction that does not fulfill all the requirements of the three-dimensional Radon transform is acceptable, then a conventional parallel rebinned convolution backprojection can be employed. A parallel rebinning processor 34 rebins the filtered projection data into parallel views. A ramp convolution processor 36 performs one-dimensional convolution filtering to generate filtered projections. A three-dimensional parallel backprojector processor 42 receives the rebinned data and performs a three-dimensional parallel backprojection to obtain a reconstructed image representation that is stored in an image memory 44.

Optionally, the filtered backprojector processor 42 includes weighting of the projection data with respect to detector position (aperture weighting) to reduce artifacts introduced by large projection components in the cone angle direction. The filtered backprojector processor 42 can also include weighting of the projection data with respect to the helix angle (angular weighting) to smooth the data at angular discontinuities, to combine angularly complementary projection data, or the like. Suitable three-dimensional filtered backprojector pipes are described, for example, in U.S. Pat. No. 6,104,775 issued to Tuy, and in U.S. patent application Ser. No. 10/274,816 by Heuscher et al. filed on Oct. 21, 2002.

The resultant image representation is suitably processed by a video processor 50 to generate a three-dimensional rendering, one or more image slices, or other visual representation of the reconstructed image that is displayed on a user interface 52. Rather than a video display, the image representation can be formatted by a printer driver and printed out using a printer, transmitted over an electronic network, stored electronically, or otherwise utilized. Preferably, the user interface 52 communicates with a computed tomography controller 54 to enable a radiologist or other operator to initiate imaging or otherwise control operation of the computed tomography scanner 10.

The inexact image reconstruction processing employing the parallel rebinning processor 34, the ramp convolution processor 36, and the parallel three-dimensional backprojector processor 42 may be adequate for certain applications. However, if an exact reconstruction which fulfills all the requirements of the three-dimensional Radon transform is desired, then an exact filtered backprojection conebeam reconstruction process with a one-dimensional shift-invariant filtering is employed. The disclosed exact reconstruction method is compatible with projection data that is axially or z-truncated. When performing exact conebeam reconstruction, a different processing path starting at a finite derivative processor 60 is engaged. That is, rather than inputting the projection data into the parallel rebinning processor 34, the data is input into the finite derivative processor 60.

With continuing reference to FIG. 1, the one-dimensional shift-invariant filtering is performed by a hybrid convolution. A finite derivative processor 60 receives conebeam projection data from the projection data memory 30 and computes derivatives with respect to the helix angle λ at fixed projection directions. A cone angle length correction processor 62 scales or normalizes lengths of the conebeam projections with respect to the source-to-detector distance D. The projection lengths vary across the curved detector 16_{c} of FIG. 2 and the flat detector 16_{f} of FIG. 3. Filtered projection data is computed from the differentiated and length-normalized projection data using a one-dimensional convolution processor 64. In a preferred embodiment, the convolution processor 64 includes a forward height rebinning processor 70, an FFT convolution processor 72 that performs a one-imensional Hilbert-based FFT convolution along the fan direction a (in the curved detector geometry) or u (in the flat detector geometry), and a reverse-height rebinning processor 74 that performs projection rebinning in the a or u direction.

A post-cosine weighting processor 80 performs a cos(α) weighting in the case of the curved detector geometry of FIG. 2. For the flat detector geometry of FIG. 3, the cos(α) weighting is preferably omitted. Omission of the cos(α) weighting in the case of a flat detector geometry is optionally achieved by setting the weights applied by the weighting processor 80 to unity. The data output by the post-cosine weighting processor 80 in the case of a curved detector geometry, or output by the convolution processor 64 in the case of a flat detector geometry, is suitably input to a conebeam backprojection processor 82 which backprojects the filtered projection data to generate an image representation that is stored in the image memory 44.

The image representation is suitably processed by the video processor 50 and displayed on the user interface 52, or otherwise utilized.

Alternatively, the data output by the convolution processor 64 is input to an inverse cosine weighting processor 80′, parallel-rebinned by a parallel rebinning processor 34′, and backprojected by the 3D parallel backprojection processor 42 to generate an image representation that is stored in the image memory 44. For the flat detector geometry of FIG. 3, the inverse cosine weighting is suitably omitted. It will be appreciated that this alternative processing of the hybrid-convolved data advantageously can employ the same 3D parallel backprojection processor 42 used in the inexact reconstruction. Moreover, the parallel rebinning processor 34′ optionally employs the same physical hardware or software module as the parallel rebinning processor 34 of the inexact reconstruction.

Those skilled in the art will appreciate that the described hybrid convolution processing operates entirely within the native scan coordinates of the curved or flat detector 16_{c}, 16_{f}. This increases reconstruction speed versus exact reconstruction performed in a detector independent coordinate system by eliminating computationally intensive rebinning operations associated with converting to the detector-independent voxel-based geometry. Moreover, reconstruction accuracy is promoted by avoiding interpolations involved in the rebinning to a voxel-based coordinate system.

The reconstruction processing components can be physically implemented in various ways. Some or all components can be software modules that are executed on one or more computers. Some or all components can be application-specific hardware pipeline components. Some or all of the reconstruction processing components can be integrated into the user interface 52, and/or into the computed tomography scanner 10, or can be stand-alone components. Hardware pipeline components are readily embodied as computer cards that insert into interface slots of a computer to enable integration of hardware- and software-based reconstruction processing components. Moreover, a given component, such as the backprojector 42, 82 can be implemented partially in software and partially as a hardware pipeline. Those skilled in the art can readily construct other physical implementations using other combinations of hardware and software.

With reference to FIG. 4, π-line and κ-plane geometrical constructs that are used in the exact conebeam reconstruction are described. A π-line is a line segment that connects two vertex points a(λ) separated by less than one pitch P in the axial or z-direction. (Equation (1) defines the pitch P). FIG. 4 shows a helical trajectory H of the x-ray source 12 and a vertex point a(λ) of interest. All π-lines have the following property: for every point within the field of view, there is a unique π-line that passes through that point. Geometrically, a helix segment connecting the endpoints of a π-line define a 180° angular coverage for points along the π-line within the field of view. Axially truncated conebeam data acquired along a helix segment joining the endpoints of a kline provide enough information for exact reconstruction of image points along the π-line.

A κ-plane is a plane that has three intersections with the helix such that the middle helix intersection point is equally angularly spaced in helix angle λ from the two other helix intersection points. There exists a family of K,-planes corresponding to each vertex point a(λ). These corresponding κ-planes are indexed by an angle W that lies in a range (−π, π). The κ-plane of angle ψ at the vertex point a(λ) is designated herein as K(λ,ψ) and contains the vertex points a(λ), a(λ+ψ), and a(λ+2ψ). A unit vector normal to K(λ,ψ) and having an acute angle with respect to the helix axis is designated herein as n(λ,ψ), and is given by:

$$\underline{n} ( λ , ψ ) = \frac{\underline{a} ( λ + ψ ) - \underline{a} ( λ ) \times \underline{a} ( λ + 2 ψ ) - \underline{a} ( λ )}{‖ \underline{a} ( λ + ψ ) - \underline{a} ( λ ) \times \underline{a} ( λ + 2 ψ ) - \underline{a} ( λ ) ‖} sign ( ψ ) \qquad ( 2 )$$

where the symbol “x” denotes a cross-product. As the angle ψ tends toward zero, the κ-plane K(λk,ψ) converges to a plane that is tangent to the helix H at a(λ) and is parallel to the detector v coordinate, that is:

$${lim}_{ψ \to 0} \underline{n} ( λ , ψ ) = \frac{1}{\sqrt{1 + 4 {π}^{2} {R}_{o}^{2} / {P}^{2}}} [ sin ( λ + {λ}_{0} ) , - cos ( λ + {λ}_{0} ) , 2 π {R}_{o} / P ] . \qquad ( 3 )$$

All κ-planes have the following property, described with reference to FIG. 4. A plane S is defined that intersects the field of view, is parallel to the helix axis, and contains a(λ). The plane S also contains two π-lines that connect the vertex point a(λ) with two other vertex points on the helix that also lie within the plane S. The κ-planes have the property that for any line L that contains a(λ) and that lies in the plane S between the two a(λ)-terminating π-lines lying in the plane S, there exists a value of the angle ψ in the range (−π, π) at which the line L belongs to the κ-plane K(λ,ψ). This property holds for any field of view less than the source distance Rₒ. There may be more than one κ-plane containing the line L.

With returning reference to FIG. 1, the exact conebeam reconstruction method is described in the source-focused curved detector coordinates geometry of FIG. 2. The finite derivative processor 60 computes derivative projection data g₁(λ,α,w)=g′(λ,θ_{c}) with respect to helix angle λ at fixed projection direction vector θ_{c}. For the curved detector coordinate system, rotated coordinate axes [eᵤ(λ), eᵥ(λX), e_{w}] are defined with respect to the stationary coordinates (x, y, z) in terms of the helix angle λ according to:
eᵤ(λ)=[−sin(λ+λₒ), cos(λ+λₒ), 0]eᵥ(λ)=[−cos(λ+λₒ), −sin(λ+λₒ), 0]e_{w}=[0, 0, 1]
where the directions u, v, w are set forth in Table I. In this rotated coordinate system and for the curved detector geometry of FIG. 2, the projection direction vector θ_{c} is given by:

$${\underline{θ}}_{c} = \frac{1}{\sqrt{{D}^{2} + {w}^{2}}} ( D sin ( α ) {\underline{e}}_{u} ( λ ) + D cos ( α ) {\underline{e}}_{v} ( λ ) + w {\underline{e}}_{w} ) . \qquad ( 5 )$$

Conversely, given the projection direction vector θ_{c} the curved detector coordinates (α,w) can be computed as:

$$\begin{matrix}α = {tan}^{- 1} ( \frac{{\underline{θ}}_{c} \cdot {\underline{e}}_{u} ( λ )}{{\underline{θ}}_{c} \cdot {\underline{e}}_{v} ( λ )} ) , & & w = \frac{D ( {\underline{θ}}_{c} \cdot {\underline{e}}_{w} )}{\sqrt{1 - {( {\underline{θ}}_{c} \cdot {\underline{e}}_{w} )}^{2}}} ,\end{matrix} \qquad ( 6 )$$

where the dot operator denotes a dot product. Using these coordinates, the derivative of g(λ,α,w) with respect to helix angle λ at fixed projection direction θ_{c} for the curved detector coordinate system is given by:

$${g}_{1} ( λ , α , w ) = {g}^{′} ( λ , {\underline{θ}}_{c} ) = {lim}_{ɛ \to 0} \frac{g ( λ + ɛ , {\underline{θ}}_{c} ) - g ( λ , {\underline{θ}}_{c} )}{ɛ} . \qquad ( 7 )$$

The derivative of Equation (7) is preferably implemented by the finite derivative processor 60 using a discrete finite difference derivative computation that arithmetically combines four neighboring projection samples. For an exemplary case where the sampling increment in coordinate α is one-fourth the sampling increment in the helix angle λ, that is, Δλ=4Δα, a suitable finite derivative is:
D′ₙⱼ=a₀Dₙ₋₁ⱼ+a₁Dₙⱼ+a₂Dₙⱼ₊₁+a₃Dₙ₊₁ⱼ₊₁
where n is the discrete projection sample index along the helix angle direction λ, j is the discrete projection sample index along the a coordinate direction, and the constants a₀, a₁, a₂, a₃ are given by:
a₀=⅛
a₁=⅞
a₂=−⅞
a₃=−⅛
A half-sample shift along the helix angle λ is introduced by the finite derivative of Equations (8) and (9).

It will be recognized that the finite derivative of Equation (8) is advantageously performed on the projection data in the curved detector coordinate system without computationally intensive rebinning. Equations (8) and (9) are exemplary equations for the specific case of finite derivative computation using four neighboring projections with a spacing ratio of Δλ=4Δα. Those skilled in the art can readily construct other finite derivatives using another number of neighboring projections, for other spacing ratios, and so forth.

With reference to FIG. 5, the differentiated projection data g₁(λ,α, w)=g′(λ,θ_{c}) output by the finite derivative processor 60 is processed by the cone angle length correction processor 62. The length of a projection g_{(c)}(λ,α,w) for a given coordinate w in the cone angle direction is independent of the fan coordinate α, due to the source-focused curvature of the detector 16_{c} along fan direction. However, as shown in FIG. 5, a projection g_{(c)}(λ,α,w) has a projection length (D²+w²)^{0.5}. Accordingly, projections g_{(c)}(λ,α,0) have projection length D. Hence, the projection lengths for the source-focused curved detector 16_{c} are suitably normalized to the source-to-detector distance D by the operation:

$${g}_{2} ( λ , α , w ) = cos ( β ) {g}_{1} ( λ , α , w ) = \frac{D}{\sqrt{{D}^{2} + {w}^{2}}} {g}_{1} ( λ , α , w ) . \qquad ( 10 )$$

The differentiated and length-normalized projection data output by the cone angle length correction processor 62 is processed by the convolution processor 64. Preferably, the convolution is implemented by the FFT convolution processor 72. The convolution is a one-dimensional convolution with respect to coordinate a performed along intersections of κ-planes K(λ,ψ) with the curved detector 16_{c}. In other words, the convolution is a one-dimensional convolution with respect to coordinate a performed at a fixed-angle ψ. Hence, prior to input to the FFT convolution processor 72, the differentiated and length-normalized projection data is rebinned by the forward height rebinning processor 70 to get constant ψ surfaces according to:
g₃(λ,α,ψ)=g₂(λ,α,w_{κ}(α,ψ)) (11)
over all ψ in a range [−π/2-αₘ, π/2+αₘ] where αₘ is a fan angle defined by the size R of the field of view and the helix radius Rₒ, that is, αₘ=arcsin(R/Rₒ), with w_{κ}(α,ψ) given by:

$${w}_{κ} ( α , ψ ) = \frac{DP}{2 π {R}_{o}} ( ψ cos ( α ) + \frac{ψ}{tan ( ψ )} sin ( α ) ) . \qquad ( 12 )$$

At a fixed angle ψ, it will be recognized that Equation (12) describes a curve in the detector area that is the intersection between the detector array of the curved detector 16_{c} and the κ-plane K(λ,ψ). This curve is referred to herein as a κ-curve of angle ψ.

With reference to FIG. 6, exemplary κ-curves for a source-focused curved detector panel with sixteen detector rows is shown. In FIG. 6 the solid lines are the κ-curves, and a Tam-Danielsson window is shown as a shaded area. The Tam-Danielsson window will be referenced later.

The FFT convolution processor 72 is applied to the rebinned data g₃(λ,α,ψ) as a one-dimensional convolution in angle α for constant angle ψ according to:

$${g}_{4} ( λ , α , ψ ) = {\int }_{- π / 2}^{+ π / 2} ⅆ {α}^{′} {h}_{H} ( sin ( α - {α}^{′} ) ) {g}_{3} ( λ , {α}^{′} , ψ ) \qquad ( 13 )$$

where h_{H} is a kernel based on a Hilbert transform. A suitable kernel h_{H} is abs(sin(x))/sin(x) which in the s-domain is:

$${h}_{H} ( s ) = - {\int }_{- ∞}^{+ ∞} ⅆ σ ⅈ sgn ( σ ) {ⅇ}^{ⅈ 2 π σ s} = \frac{1}{π s} . \qquad ( 14 )$$

However, in view of the half-sample shift introduced by the selected finite derivative of Equations (8) and (9), a compensating half-sample shift is preferably incorporated into the kernel h_{H}.

After the one-dimensional convolution processor 72, the projection data g₄(λ,α,ψ) is processed by the backward height rebinning processor 74 to obtain rebinned projection data g₅(λ,α,w) according to:
g₅(λ,α,w)=g₄(λ,α,ψₘᵢₙ(α,w)) (15)
where ψₘᵢₙ is the angle ψ of smallest absolute value that satisfies the equation:

$$w = \frac{DP}{2 π R} ( ψ cos ( α ) + \frac{ψ}{tan ( ψ )} sin ( α ) ) . \qquad ( 16 )$$

Reviewing the operation of the exemplary convolution processor 64, it will be seen that the output g₅(λ,α,w) at a given detector location (αₒ,w₀) is obtained by convolving projection data g₂(λ,α,w) on the κ-curve of smallest absolute ψ value that passes through (αₒ,wₒ).

For processing data in the source-focused curved detector geometry, the output of the convolution processor 64 is preferably processed by the post-cosine weighting processor 80. This weighting adjusts for the convolution in the fan direction according to:
g^{F}_{(c)}(λ,α,w)=cos(α)g₅(λ,α,w) (17)
to produce the final filtered data g^{F}_{(c)}(λ,α,w).

The filtered data g^{F}_{(c)} is suitable for subsequent processing by a conebeam backprojector processor 82 to produce an exact reconstruction of conebeam projection data. In a suitable embodiment, the conebeam backprojector processor 82 computes the image f(x) according to:

$$f ( \underline{x} ) = \frac{1}{2 π} {\int }_{{λ}_{i} ( \underline{x} )}^{{λ}_{o} ( \underline{x} )} ⅆ λ \frac{1}{{v}^{*} ( λ , \underline{x} )} {g}_{( c )}^{F} ( λ , {α}^{*} ( λ , \underline{x} ) , {w}^{*} ( λ , \underline{x} ) ) , \qquad ( 18 )$$

where λᵢ(x) and λₒ(x) are the endpoints of the π-line through x with λᵢ(x)<λₒ(x), and:

$$\begin{matrix}{v}^{*} ( λ , \underline{x} ) = R - x cos ( λ + {λ}_{o} ) - y sin ( λ + {λ}_{o} ) , & ( 19 ) \\ {α}^{*} ( λ , \underline{x} ) = arctan ( \frac{1}{{v}^{*} ( λ , \underline{x} )} ( - x sin ( λ + {λ}_{o} ) + y cos ( λ + {λ}_{o} ) ) ) , & ( 20 ) \\ and & \\ {w}^{*} ( λ , \underline{x} ) = \frac{D cos ( {α}^{*} ( λ , \underline{x} ) )}{{v}^{*} ( λ , \underline{x} )} ( z - {z}_{o} - \frac{P}{2 π} ( λ + {λ}_{o} ) ) . & ( 21 )\end{matrix}$$

The backprojection of Equations (18)-(21) implemented by the conebeam backprojector processor 82 is exemplary only. Those skilled in the art can employ other suitable backprojection processes. For instance, in an alternative backprojection path shown in FIG. 1, the filtered projection data g^{F}_{(c)} is input to the inverse cosine weighting processor 80′, the parallel rebinning processor 34′, and the parallel-rebinned filtered projection data is input to the 3D parallel backprojector 42 for backprojection. This latter pathway advantageously eliminates the computationally intensive 1/v weighting of the conebeam backprojector 82. The inverse cosine weighting processor 80′ applies a 1/cos(α) weighting. This different weighting as compared with the cos(α) weighting applied by the cosine weighting processor 80 (see Equation 17) accounts for a difference in internal projection data weighting in the two backprojectors 42, 80. A suitable 3D parallel backprojector 42 is described in U.S. patent application Ser. No. 10/274,816 by Heuscher et al., filed on Oct. 21, 2002, which discloses a three-dimensional parallel backprojector pipeline that advantageously facilitates weighted combining of redundant projection data, that is, projection data for a given voxel that are separated in helix angle by integer multiples of 180°, to compensate for motion artifacts and other imaging imperfections.

In performing above-described exact reconstruction method, the number of detector rows should be large enough so that the filtered data can be computed for points of the region of interest. The number of detector rows, the helix pitch P, the helix radius Rₒ, and the size of the field of view can be interrelated. One example of a suitable detector region for conebeam reconstruction by the backprojection of Equation (18) is known in the art as the Tam-Danielsson window. An exemplary Tam-Danielsson window is shown as the shaded area on the detector in FIG. 6. Referring again to the angle αₘ, where αₘ is the fan angle defined by the size R of the field of view and the helix radius Rₒ, that is, αₘ=arcsin(R/Rₒ), the region of the Tam-Danielsson window is the set of (α,w) points such that a lies in a range [−αₘ,αₘ], and w lies between a minimum value w_{bottom}(α) given by:

$${w}_{bottom} ( α ) = - \frac{D P}{2 π {R}_{o}} \frac{π / 2 + α}{cos ( α )} \qquad ( 22 )$$

and a maximum value wwp(a) given by:

$${w}_{top} ( α ) = \frac{DP}{2 π {R}_{o}} \frac{π / 2 - α}{cos ( α )} . \qquad ( 23 )$$

The convolution processor 64 is a non-local process that uses projection data outside the Tam-Danielsson window. However, as shown in FIG. 6 the number of detector rows needed to cover the Tam-Danielsson window generally also covers the range between the κ-curves K(λ,ψ) in the range −π/2-αₘ≦ψ≦π/2+αₘ. Hence, it is generally sufficient for the exact conebeam reconstruction to provide enough detector rows to span the range [w_{bottom},wₜₒₚ]. This number of detector rows is about:

$${N}_{rows} = \frac{{d}_{w} + {w}_{top} ( {α}_{m} ) - {w}_{bottom} ( {α}_{m} )}{{d}_{w}} = 1 + \frac{DP}{π {R}_{o} {d}_{w}} \frac{π / 2 + {α}_{m}}{cos ( {α}_{m} )} , \qquad ( 24 )$$

where d_{w} is a thickness of the detector rows.

The exact conebeam reconstruction method described with reference to Equation (4) through Equation (24) is suitable for reconstructing projection data formatted in the source-centered curved detector geometry of FIG. 2. Reconstruction of projection data formatted in the flat detector geometry of FIG. 3 is described next.

With reference to FIGS. 1 and 3, the flat detector array 16_{f} is parallel to the helix axis, that is, parallel to the z-direction. The orientation of the detector array 16_{f} changes with helical movement of the vertex point a(λ), that is, with the helix angle λ, so as to remain orthogonal to a plane containing both the vertex point a(λ) and the helix axis. As shown in FIG. 1, this is suitably accomplished by mounting the detector 16_{f} opposite the x-ray source 12 on the rotating gantry 22. Alternatively, the flat detector can be mounted as a detector ring on the stationary gantry 24. Similarly to the case of the curved detector, the source-to-detector distance D corresponds to a distance from the vertex point a(λ) to the plane of the flat detector 16_{f}, as indicated in FIG. 3. The rotated coordinate axes [eᵤ, eᵥ, e_{w}] of Equation (4) apply to the flat detector geometry as well.

The projection direction vector θ_{f} for the flat detector geometry is given by:

$${\underline{θ}}_{f} = \frac{1}{\sqrt{{u}^{2} + {D}^{2} + {w}^{2}}} ( u {\underline{e}}_{u} ( λ ) + D {\underline{e}}_{v} ( λ ) + w {\underline{e}}_{w} ) . \qquad ( 25 )$$

Conversely, given the projection direction vector θ_{f} the flat detector coordinates (u,w) can be computed as:

$$\begin{matrix}u = D \frac{{\underline{θ}}_{f} \cdot {\underline{e}}_{u} ( λ )}{{\underline{θ}}_{f} \cdot {\underline{e}}_{v} ( λ )} , & & w = D \frac{{\underline{θ}}_{f} \cdot {\underline{e}}_{w}}{{\underline{θ}}_{f} \cdot {\underline{e}}_{v} ( λ )} ,\end{matrix} \qquad ( 26 )$$

where the dot operator denotes a dot product. Using these coordinates, the derivative of projection data g(λ,u,w) for the flat detector coordinate system with respect to helix angle λ at fixed projection direction θ_{f} is computed by the finite derivative processor 60 in accordance with Equations (7)-(9), making the substitutions of θ_{f} for θ_{c} and u for α in Equation (7), to obtain differentiated projection data g₁(λ,u,w)=g′(λ,θ_{f}).

The length correction in the case of the flat detector geometry is computed by the cone angle length correction processor 62 as:

$${g}_{2} ( λ , u , w ) = \frac{D}{\sqrt{{u}^{2} + {D}^{2} + {w}^{2}}} {g}_{1} ( λ , u , w ) , \qquad ( 27 )$$

which is analogous to the normalization of Equation (10) for the curved detector geometry.

The forward height rebinng processor 70 performs rebinning of the flat detector geometry projection data according to:
g₃(λ,u,ψ)=g₂(λ,u,w_{κ}(u,ψ)) (28),
where w_{κ}(u,ψ) is:

$${w}_{κ} ( u , ψ ) = \frac{D P}{2 π {R}_{o}} ( ψ + \frac{ψ}{tan ( ψ )} \frac{u}{D} ) . \qquad ( 29 )$$

Equations (28) and (29) are analogous to Equations (11) and (12) of the curved detector geometry processing. Analogously to the case of the curved detector geometry, Equation (29) defines κ-curves K(λ,ψ) in the flat detector geometry that correspond to intersections of κ-planes with the flat detector 16_{f}.

The one-dimensional convolution processor 72 performs the following one-dimensional convolution with respect to coordinate u at constant angle ψ:

$${g}_{4} ( λ , u , ψ ) = {\int }_{- ∞}^{+ ∞} ⅆ {u}^{′} {h}_{H} ( u - {u}^{′} ) {g}_{3} ( λ , {u}^{′} , ψ ) \qquad ( 30 )$$

where a suitable kernel h_{H} is given in Equation (14). The reverse height rebinning processor 74 rebins the convolved projection data g₄(λ,u,ψ) to produce filtered projection data g^{F}_{(f)}(λ,u,w) according to:
g^{F}(λ,u,w)=g₄(λ,u,ψₘᵢₙ(u,w)) (31),
where ψₘᵢₙ is the angle of smallest absolute value that satisfies:

$$w = \frac{D P}{2 π R} ( ψ + \frac{ψ}{tan ( ψ )} \frac{u}{D} ) . \qquad ( 32 )$$

For the flat detector geometry, the post cosine weighting processor 80 or the inverse cosine weighting processor 80′ is bypassed, and so the output of the reverse height rebinning processor 74 is the filtered projection data g^{F}_{(f)}(λ,u,w). It will be appreciated that the processes set forth in Equations (28)-(32) obtain g^{F}_{(f)}(λ,uₒ,wₒ) from the projection data g₂(λ,u,w) by convolving along the κ-curve of smallest absolute angle ψ that passes through (uₒ,wₒ).

The filtered data g^{F}_{(f)} is suitable for subsequent processing by the conebeam backprojector processor 82 to produce an exact reconstruction of conebeam projection data. In a suitable embodiment, the conebeam backprojector processor 82 computes the image f(x) according to:

$$f ( \underline{x} ) = \frac{1}{2 π} {\int }_{{λ}_{i} ( \underline{x} )}^{{λ}_{o} ( \underline{x} )} ⅆ λ \frac{1}{{v}^{*} ( λ , \underline{x} )} {g}_{( f )}^{F} ( λ , {u}^{*} ( λ , \underline{x} ) , {w}^{*} ( λ , \underline{x} ) ) , \qquad ( 33 )$$

analogous to the backprojecting of the filtered projection data g^{F}_{(c)} of the curved coordinate geometry given in Equation (18). The parameter v*(λ,x) is the same as that given in Equation (19) for the curved detector geometry, while the parameters u*(λ,x) and w*(λ,x) are given in the flat detector geometry by:

$$\begin{matrix}{u}^{*} ( λ , \underline{x} ) = \frac{D}{{v}^{*} ( λ , \underline{x} )} ( - x sin ( λ + {λ}_{o} ) + y cos ( λ + {λ}_{o} ) ) , & ( 34 ) \\ and & \\ {w}^{*} ( λ , \underline{x} ) = \frac{D}{{v}^{*} ( λ , \underline{x} )} ( z - {z}_{o} - \frac{P}{2 π} ( λ + {λ}_{o} ) ) . & ( 35 )\end{matrix}$$

As in the case of backprojecting the filtered projection data g^{F}_{(c)} of the curved coordinate geometry, the backprojection process of Equations (19) and (33)-(35) is exemplary only. In one alternative backprojection approach, the filtered backprojection data g^{F}_{(f)} is input into the parallel rebinning processor 34′, and the parallel-rebinned filtered backprojection data is backprojected using the 3D parallel backprojector 42.

In the case of the flat detector geometry, a suitable number of detector rows N_{rows} is estimated as follows. The Tam-Danielsson window is the set of (u,w) points with u in a range [−uₘ,uₘ] where uₘ=D tan(αₘ)=D tan(arcsin(R/Rₒ)), and w in a range [w_{bottom}(u),wₜₒₚ(u)], where:

$$\begin{matrix}{w}_{bottom} ( u ) = - \frac{P}{2 π {R}_{o} D} ( {u}^{2} + {D}^{2} ) ( π / 2 + arctan ( \frac{u}{D} ) ) , & ( 36 ) \\ and & \\ {w}_{top} ( u ) = - \frac{P}{2 π {R}_{o} D} ( {u}^{2} + {D}^{2} ) ( π / 2 - arctan ( \frac{u}{D} ) ) . & ( 37 )\end{matrix}$$

Computation of the filtered data at any point in the Tam-Danielsson window employs data on κ-curves that intersect the Tam-Danielsson window. This data generally corresponds to data acquired using detector rows between u_{bottom} and uₜₒₚ inclusive, and so a suitable number of detector rows is about:

$${N}_{rows} = \frac{{d}_{w} + {w}_{top} ( - {u}_{m} ) - {w}_{bottom} ( - {u}_{m} )}{{d}_{w}} , \qquad ( 38 )$$

which can be rewritten as:

$${N}_{rows} = 1 + \frac{P}{π {R}_{o} D {d}_{w}} ( {u}_{m}^{2} + {D}^{2} ) ( \frac{π}{2} + arctan ( \frac{{u}_{m}}{D} ) ) , \qquad ( 39 )$$

where d_{w} is the thiclkess of the detector rows.

The exact nature of the exact reconstructions described herein has been demonstrated by showing equivalency of image representations reconstructed using the techniques disclosed herein and image representations reconstructed using the exact method of Katsevich. The exact reconstruction methods described herein advantageously are performed entirely within the flat or curved detector coordinate system. Computationally intensive transformations of projection data into a detector-independent coordinate system is thus obviated, providing a more rapid reconstruction. The exact reconstruction methods described herein optionally incorporate the three-dimensional backprojector 42. This enables the exact reconstruction to take advantage of three-dimensional backprojector capabilities such as complementary combination of redundant projection data..

The invention has been described with reference to the preferred embodinents. Obviously, modifications and alterations will occur to others upon reading and understanding the preceding detailed description. It is intended that the invention be construed as including all such modifications and alterations insofar as they come within the scope of the appended claims or the equivalents thereof.

1. A conebeam computed tomography imaging system including:
an x-ray source that produces an x-ray conebeam directed into an examination region, the x-ray source being arranged to traverse a generally helical trajectory around the examination region;
an x-ray detector array arranged to detect the x-ray conebeam after passing through the examination region, the x-ray detector array generating projection data in native scan coordinates defmed with reference to the detector array; and
an exact reconstruction processor that performs an exact reconstructing of conebeam projection data produced by the detector array into an image representation, the reconstructing being performed in the native scan coordinates.

2. The conebeam computed tomography imaging system as set forth in claim 1, wherein the reconstruction processor includes:
a derivative processor that computes a derivative of the projection data with respect to a helix angle of the helical trajectory at fixed projection direction to generate differentiated projection data;
a convolution processor that convolves the differentiated projection data with a kernel function to produce filtered projection data, the convolving being performed in the native scan coordinates; and
a backprojector that backprojects the filtered projection data to obtain an image representation.

3. The conebeam computed tomography imaging system as set forth in claim 2, wherein the convolving performed by the convolution processor is a one-dimensional convolving employing a kernel function based on a Hilbert transform.

4. The conebeam computed tomography imaging system as set forth in claim 2, wherein the derivative processor computes the derivative using a discrete finite difference derivative computation that arithmetically combines selected neighboring projection data.

5. The conebeam computed tomography imaging system as set forth in claim 2, wherein the reconstruction processor further includes:
a cone angle length correction processor that normalizes a length of each projection to a source-to-detector distanced.

6. The conebeam computed tomography imaging system as set forth in claim 2, wherein the reconstruction processor further includes:
a parallel rebinning processor that parallel-rebins the filtered projection data, the backprojector being a parallel backprojector that backprojects the parallel rebinned filtered backprojection data.

7. The conebeam computed tomography imaging system as set forth in claim 2, wherein the detector array has a curved detector geometry generally focused on the x-ray source, and the detector coordinate system is a curved detector coordinate system having native scan coordinates including a helix angle coordinate, a projection fan coordinates, and a projection cone coordinates.

8. The conebeam computed tomography imaging system as set forth in claim 7, wherein the convolution processor includes:
a first rebinning processor that rebins the differentiated projection data along K-curves to produce y-rebinned projection data where ψ indicates the κ-curve; and
a one-dimensional convolution transform processor that one-dimensionally convolves the y-rebinned projection data with the kernel function to produce convolved projection data, the one dimensional convolving being in the projection fan coordinate at constant ψ.

9. The conebeam computed tomography imaging system as set forth in claim 8, wherein the one dimensional convolution transform processor perforns the one-dimensional convolution according to:
$${g}_{conv} ( λ , α , ψ ) = {\int }_{- π / 2}^{+ π / 2} ⅆ {α}^{′} {h}_{H} ( sin ( α - {α}^{′} ) ) {g}_{rebin1} ( λ , {α}^{′} , ψ )$$
where g_{rebin1}(λ,α,ψ) indicates the first rebinned data, h_{H}( ) indicates the kernel function, and g_{conv}(λ,α,ψ) indicates the convolved projection data.

10. The conebeam computed tomography imaging system as set forth in claim 8, wherein the convolution processor further includes:
a weighting processor that weights the convolved projection data by one of a cosine of the projection fan coordinate and an inverse cosine of the projection fan coordinate to produce the filtered projection data.

11. The conebeam computed tomography imaging system as set forth in claim 10, wherein the backprojector includes:
a conebeam backprojector that applies a 1/v weighting to the filtered projection data during the backprojecting.

12. The conebeam computed tomography imaging system as set forth in claim 7, wherein the reconstruction processor further includes:
a cone angle length correction processor that scales projections by a cosine of the projection cone coordinate.

13. The conebeam computed tomography imaging system as set forth in claim 2, wherein the detector array has a substantially flat detector geometry, and the detector coordinate system is a flat detector coordinate system having native scan coordinates including a helix angle coordinate, a projection fan detector coordinate, and a projection cone coordinate.

14. The conebeam computed tomography imaging system as set forth in claim 13, wherein the convolution processor includes:
a first rebinning processor that rebins the differentiated projection data along K-curves to produce y-rebinned projection data where ψ indicates the κ-curve; and
a one-dimensional convolution transform processor that one-dimensionally convolves the y-rebinned projection data with the kernel function to produce convolved projection data, the one dimensional convolving being in the projection fan coordinate at constant ψ.

15. The conebeam computed tomography imaging system as set forth in claim 14, wherein the one dimensional convolution transform processor perfonms the one-dimensional convolution according to:
$${g}_{conv} ( λ , u , ψ ) = {\int }_{- ∞}^{+ ∞} ⅆ {u}^{′} {h}_{H} ( sin ( u - {u}^{′} ) ) {g}_{rebin1} ( λ , {u}^{′} , ψ )$$
where g_{rebin1}(λ,u,ψ) indicates the first rebinned data, h_{H}( ) indicates the kernel function, and g_{conv}(λ,u,ψ) indicates the convolved projection data.

16. The conebeam computed tomography imaging system as set forth in claim 14, wherein the convolution processor further includes:
a reverse height rebinning processor that rebins the convolved projection data.

17. The conebeam computed tomography imaging system as set forth in claim 1, wherein the detector array has a curved detector geometry generally focused on the x-ray source, and the detector coordinate system is a curved detector coordinate system having native scan coordinates including a helix angle coordinate, a projection fan coordinate, and a projection cone coordinate.

18. The conebeam computed tomography imaging system as set forth in claim 1, wherein the detector array has a substantially flat detector geometry, and the detector coordinate system is a flat detector coordinate system having native scan coordinates including a helix angle coordinate, a projection fan detector coordinate, and a projection cone coordinate.

19. The conebeam computed tomography imaging system as set forth in claim 1, wherein the reconstruction processor includes:
a finite derivative processor that computes a finite derivative of the projection data in the native scan coordinates along a first direction to generate differentiated projection data;
a convolution processor that performs a one-dimensional convolution of the differentiated projection data in the native scan coordinates along a second direction that is different from the first direction to produce filtered projection data; and
a backprojector that backprojects the filtered projection data to obtain an image representation.

20. A conebeam computed tomography imaging system that produces conebeam computed tomography projection data having native scan coordinates that include at least a helix angle, a projection fan coordinate and a projection cone coordinate, the imaging system including:
a means for computing filtered projection data, the means for computing including a differentiating means for computing a derivative of projection data with respect to the helix angle at fixed projection direction and a convolving means for convolving projection data with a kernel function, the convolving being performed in the native scan coordinates; and
a means for backprojecting the filtered projection data to obtain an image representation.

21. An exact reconstruction method for reconstructing conebeam computed tomography projection data having native scan coordinates that include at least a helix angle, a projection fan coordinate, and a projection cone coordinate, the method including:
computing filtered projection data by a combination of:
computing a derivative of projection data with respect to the helix angle at fixed projection direction, and
convolving projection data with a kernel function, the convolving being performed in the native scan coordinates; and
backprojecting the filtered projection data to obtain an image representation.

22. The method as set forth in claim 21, wherein the computing of a derivative includes:
computing each differentiated projection datum by a finite difference derivative based on a plurality of projection data that neighbor said differentiated projection datum.

23. The method as set forth in claim 21, wherein the convolving includes:
rebinning projection data with respect to κ-planes K to group projections of the projection data by κ-plane coordinate ψ; and
one-dimensionally convolving the rebinned projection data with the kernel function, the one-dimensional convolving being with respect to the projection fan coordinate, the one-dimensional convolving being at fixed κ-plane coordinate ψ.

24. The method as set forth in claim 21, wherein the backprojecting includes:
parallel rebinning the filtered projection data to obtain parallel projection views; and
backprojecting the parallel projection views.

25. The method as set forth in claim 24, wherein the native scan coordinates have a curved geometry, and the method further includes:
prior to the backprojecting, weighting the filtered projection data by an inverse cosine of the projection fan coordinate.

26. The method as set forth in claim 21, wherein the backprojecting includes:
weighting the filtered projection data by a 1/v factor; and
backprojecting the filtered data with the 1/v weighting.

27. The method as set forth in claim 26, wherein the native scan coordinates have a curved geometry, and the method further includes:
prior to the backprojecting, weighting the filtered projection data by a cosine of the projection fan coordinate.

28. The method as set forth in claim 21, wherein the kernel function is based on a Hilbert transform.

29. The method as set forth in claim 21, wherein the computing of filtered projection data further includes:
normalizing a projection length of projection data.
